SEQUENCE LISTING (6 sequences)

SEQ ID NO: 1 - DNA, 19 bp, Artificial Sequence
  NDM-1 Forward Primer
ggtttggcgatctggtttt

SEQ ID NO: 2 - DNA, 20 bp, Artificial Sequence
  NDM-1 Reverse Primer
cggaatggctcatcacgatc

SEQ ID NO: 3 - DNA, 20 bp, Artificial Sequence
  KPC-2 Forward Primer
atgtcactgtatcgccgtct

SEQ ID NO: 4 - DNA, 20 bp, Artificial Sequence
  KPC-2 Reverse Primer
ttttcagagccttactgccc

SEQ ID NO: 5 - DNA, 20 bp, Artificial Sequence
  OXA-48 Forward Primer
gcgtggttaaggatgaacac

SEQ ID NO: 6 - DNA, 20 bp, Artificial Sequence
  OXA-48 Reverse Primer
cggttgggttgaacttgatg

(19) United States
(12) Patent Application Publication
(10) Pub. No.: US 2022/0145358 A1
(43) Pub. Date: May 12, 2022

(54) DIRECT CARBAPENEMASE TRIPLE (KPC-2, OXA-48, NDM-1) PCR DIAGNOSTIC KIT

(71) Applicant: TRAKYA UNIVERSITESI REKTORLUGU, Edirne (TR)

(72) Inventor: Sebnem BUKAVAZ, Edirne (TR)

(73) Assignee: TRAKYA UNIVERSITESI REKTORLUGU, Edirne (TR)

(21) Appl. No.: 17/429,357

(22) PCT Filed: Feb. 3, 2020
(86) PCT No.: PCT/TR2020/050066

§ 371 Date: Aug. 9, 2021

(30) Foreign Application Priority Data
Feb. 11, 2019 (TR) 2019/01998

(51) Int. Cl. C12Q 1/686; C12Q 1/34

(52) U.S. Cl. CPC C12Q 1/686; C12Q 2600/16; C12Q 1/34

(57) ABSTRACT

A PCR based rapid diagnostic kit for detection of carbapenemases such as Klebsiella pneumoniae carbapenemase (KPC), New Delhi metallo-β-lactamase (NDM), and Oxacillinase-48 (Oxa-48). The PCR based rapid diagnostic kit includes a primer pair of SEQ ID NO: 1 and SEQ ID NO: 2, a primer pair of SEQ ID NO: 3 and SEQ ID NO:4, and a primer pair of SEQ ID NO: 5 and SEQ ID NO: 6.

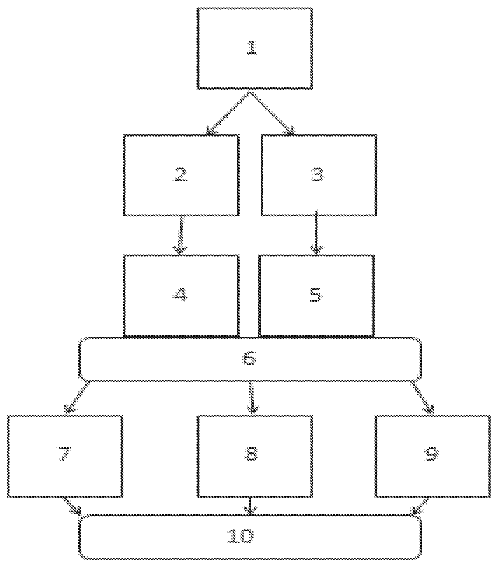

### CROSS REFERENCE TO THE RELATED APPLICATIONS

This application is the national phase entry of International Application No. PCT/TR2020/050066, filed on Feb. 3, 2020, which is based upon and claims priority to Turkish Patent Application No. 2019/01998, filed on Feb. 11, 2019, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBUY141_Sequence Listing.txt, created on Jul. 19, 2021 and is 1,464 bytes in size.

### TECHNICAL FIELD

The present invention is related to PCR based rapid detection kit of carbapenemases which are responsible for carbapenem antibiotic resistance.

### BACKGROUND

Carbapenemases are the β-lactamase enzymes that hydrolyse penicillins, most of the cephalosporins, gradually carbapenemases, and monobactams.

Infections caused by Gram-negative bacteria with extended spectrum beta-lactamase (ESBL) and produced carbapenemases cause serious treatment problems in especially hospitalized patients with comorbidity as immunosuppression.

The most identified carbapenemases in Enterobacteriaceae family in the World are Klebsiella pneumoniae carbapenemase (KPC), New Delhi metallo-β-lactamase (NDM) and Oxacillinase-48.

Carbapenems are the most extended spectrum beta-lactam antibiotics which show rapid bactericidal effect and are used widely for infections caused by both aerobic and anaerobic microorganisms.

Currently used techniques for detecting Carbapenemases are classical microbiology culture method and antibiogram.

Currently used methods in routine microbiology laboratories consist of the bacterial identification and the antibograms required for the first 24 hours of incubation for bacterial growth from clinical materials. After the incubation period, it proceeds to the automatically bacterial identification system and antibiogram that need 24 hours. The basic principle of the automatic identification systems is based on the metabolites or enzymes that are produced during bacterial growth and cause pH changes that allow bacteria identification. As a summary, the current bacterial identification system depends on the biochemical features of the bacterial growth. This method avoids completely any technical doubt of Enterobacteriaceae members and most of the other bacterial species identification, however, it needs at least 48 hours. This time is crucial for dissemination of the resistant enzymes via patients.

The secondary process mentioned above, antibiogram, is carried out by semi-automatic systems allowed by identification of the antibiotic susceptibility or resistance of bacterium. This method reveals that bacteria have resistance enzymes, but it does not provide the enzyme details. The present invention enables precise enzyme detection to take early prevention measures such as patient isolation to avoid the outbreaks by transmission from patient to patient if the enzyme is carried by a plasmid.

Additionally, the phenotypic Carba NP and Modified Hodge Test (MHT) for identification of the carbapenemases are available. MHT is reported as a reliable method for K. pneumoniae but not other Enterobacteriaceae members. MHT informs only a general carbapenemase presence, however, Carba NP test is a high reliable method for carbapenemase identification but it is expensive.

Carbapeneme resistance enzymes (CRE) are identified in our country by mostly conventional Polymerase Chain Reaction (PCR) or internationally originated kits which allow the identification of a wide range of carbapenemase. However, because of the Euro and dollar rates, these kits are costly. Another disadvantage is that the reaction is time consuming.

U.S. Pat. No. 9,914,980 B2 numbered patent is about the identification of more than 170 different type of carbapenemase genes. It has ended up being high priced and time consuming.

### SUMMARY

The aim of the invention is a diagnostic kit design for detection of carbapenemases by PCR methods.

Thanks to the present PCR based rapid diagnostic kit, the antibiotic resistance enzymes will be rapidly (1-2 hours) and specifically identified. Thus, the outbreaks will be prevented by accelerated patient's isolation if the enzyme carried by a plasmid.

It is more crucial that the dissemination resistant enzymes in the patients in an ICU having multiple comorbidities and Carbapenem Resistance Infection (CR) will be prevented by the earlier patient isolation and the effective antibiotic treatment.

The invention may be easily designed for relevance; for example, if the kit is used in routine laboratories for diagnosis, it is designed by quantitative prob-hybridization technique and conventional single, or multiplex Sybr-Green technique if it is used in the epidemiological investigations.

Since the invention kit is directed to the three most frequently detected and important carbapenemases (Kpc-2, Oxa-48, Ndm-1) in our country, the test reaction takes several hours (3-4 h) and it would be possible at a low cost.

### BRIEF DESCRIPTION OF THE DRAWINGS

FIGURE shows a workflow.

### DEFINITIONS OF THE ASPECTS/SECTIONS/PARTS FORMING THE INVENTION

1. Carbapenemase positive bacteria as Escherichia coli, Klebsiella pneumoniae
2. Clinical samples
3. Bacterial culture from clinical samples
4. Direct DNA isolation from clinical samples
5. DNA isolation from bacterial culture
6. Standardization of DNA isolation, specifity and susceptibility tests.
7. Conventional PCR (blaKPC-2, blaOXA-48, blaNDM-1)
8. Multiplex PCR
9. Sybr-Green PCR
10. DNA sequence and variant analysis

### DETAILED DESCRIPTION OF THE EMBODIMENTS

As dedicated in FIGURE, first, carbapenemase positive Escherichia coli, Klebsiella pneumoniae etc. bacteria (1) are stored at −80° C. and cultured. Secondly, direct DNA isolation of the selected clinical samples such as blood, endotracheal aspirate and sputum (2) received in routine diagnostic laboratories is conducted as advised by the Manufacturers. DNA isolation from directly clinical samples and bacterial cultures are simultaneously performed by available bacterial DNA isolation kit or heat treatment methods. The DNA samples can be stored at +4° C. for several weeks or −80° C. for six months if they are not directly used for the next step.

After the standardization of DNA isolation, the specificity and susceptibility (validation experiments) experiments will be conducted for the production of relevant kit design for the detection of carbapenemases by the techniques of Conventional single PCR (7), Multiplex PCR (8), or Sybr-Green PCR (9).

Conventional single PCR consists of one of the carbapenemases; blaKPC-2, blaOXA-48, blaNDM-1. Multiplex PCR consists of any of two or three of carbapenemases; blaKPC-2, blaOXA-48, blaNDM-1, and Prob-hybridization of single, two or triple of carbapenemases; blaKPC-2, blaOXA-48, blaNDM-1.

With this technique, special design of DNA primers is ready for use, which targets the encoding of problematic resistance enzymes detected by PCR kits and takes into consideration of the customer goals.

It shows in the table 1, the primer sets will be used to detect different carbapenemase genes.

TABLE 1
Primers will be used for the Kit

| Forward primer | Reverse primer |
|---|---|
| NDM-1 | NDM-1 |
| GGTTTGGCGATCTGGTTTT | CGGAATGGCTCATCACGATC |
| (SEQ ID NO: 1) | (SEQ ID NO: 2) |
| KPC-2 | KPC-2 |
| ATGTCACTGTATCGCCGTC | TTTTCAGAGCCTTACTGCCC |
| T (SEQ ID NO: 3) | (SEQ ID NO: 4) |
| OXA-48 | OXA-48 |
| GCGTGGTTAAGGATGAAC | CGGTTGGGTTGAACTTGATG |
| AC (SEQ ID NO: 5) | (SEQ ID NO: 6) |

The instant kits will consist of Master mix included Taq DNA polymerase and primers to target the amplification of the specific DNA parts and two small tubes containing the positive controls, and the sterile deionized water. In the process of the carbapenemase detection, the resistance enzyme targeted PCR detection kit achieves results rapidly (approximately 3-4 hours) in a Thermal Cycler. The last step of the invention is the verifying of the amplicons by DNA sequence and variant analysis (10); if the classic PCR kit is selected, the agarose gel will be run under the conditions of 1% w/v agarose gel, 100 V for 30 min. For Sybr-Green Real-Time PCR kit, the Melt-Curve analysis will be evaluated for accurate melting point. Selected samples after amplification will be proceeded to DNA sequence analysis and the results will be evaluated at the point of the nucleotide changes and the enzyme variations will be statistically reported.

We claim:

1. A PCR diagnostic kit for a detection of a carbapenem resistance enzyme, wherein
the carbapenem resistance enzyme is a carbapenemase selected from the group consisting of Klebsiella pneumoniae carbapenemase (KPC), New Delhi metallo-β-lactamase (NDM), and Oxacillinase-48 (Oxa-48), and
the PCR diagnostic kit comprises a primer pair of SEQ ID NO: 1 and SEQ ID NO: 2, a primer pair of SEQ ID NO: 3 and SEQ ID NO:4, and a primer pair of SEQ ID NO: 5 and SEQ ID NO: 6.

2. A method of producing the PCR diagnostic kit according to claim 1, comprising the following steps:
collecting carbapenemase positive Escherichia coli and Klebsiella pneumoniae bacteria, by storing the carbapenemase positive Escherichia coli and Klebsiella pneumoniae bacteria at −80° C. and culturing the carbapenemase positive Escherichia coli and Klebsiella pneumoniae bacteria;
obtaining bacterial cultures from a clinical sample, wherein the clinical sample is a blood sample, an endotracheal aspirate, or a sputum;
performing a first DNA isolation from the clinical sample and a second DNA isolation from a bacterial culture of the carbapenemase positive Escherichia coli and Klebsiella pneumoniae bacteria;
standardizing the first DNA isolation and the second DNA isolation, and performing a validation of specificity and susceptibility;
producing the PCR diagnostic kit by using a conventional single PCR, a multiplex PCR, or a Sybr-Green PCR;
performing a DNA sequence and variant analysis.
